SEQUENCE LISTING (10 sequences)

SEQ ID NO: 1 - DNA, 90 bp, Artificial Sequence
  Forward primer for gene Delta S1z-ccdB-amp Delta S1z-ccdB-F
actggtgaagagtgttggtgacacctcttttgctggtggctcttttgtgtgcactgtgtgggcgatcgctttgtttgtttttctgaatgc

SEQ ID NO: 2 - DNA, 89 bp, Artificial Sequence
  Reverse primer for gene Delta S1z-ccdB-amp Delta S1z-ccdB-R
atgtgtctgaaatggccaaacctgcgtctgctgagtgattgtgactgacagctgggtcggtgcgatcgctttgttcaaaaaaaagccag

SEQ ID NO: 3 - DNA, 77 bp, Artificial Sequence
  Forward primer for gene Delta Nz-ccdB-amp Delta Nz-ccdB-F
cggtaaagcaactggagaagcaagcgccccagcgccagtcatcaaactaggcgatcgctttgtttatttttctaaat

SEQ ID NO: 4 - DNA, 75 bp, Artificial Sequence
  Reverse primer for gene Delta Nz-ccdB-amp Delta Nz-ccdB-R
aggagaaaacagcatacgcacgtccatagtgttaagttgagaatctagctgcgatcgctttgttcaaaaaaaagc

SEQ ID NO: 5 - DNA, 79 bp, Artificial Sequence
  Forward primer for amplifying GL15-S1 GL15-S1-F
actggtaagagatgttggtaacacctcttttactagtgactcttttgtgtgcactatgtgcaaatttgttagatcttac

SEQ ID NO: 6 - DNA, 63 bp, Artificial Sequence
  Reverse primer for amplifying GL15-S1 GL15-S1-R
acaactagtaacattactactaatagaacgtctaaaacgacgtgagctattggttaacttaac

SEQ ID NO: 7 - DNA, 66 bp, Artificial Sequence
  Forward primer for amplifying GL14-S2 GL14-S2-F
acaggttgagaaccagttttatgttaagttaaccaatagctcacgtcgttttagacgttctattag

SEQ ID NO: 8 - DNA, 80 bp, Artificial Sequence
  Reverse primer for amplifying GL14-S2 GL14-S2-R
acaagtttacaccaaagcaaaattaatactattaagaaggacgtgggactttggatcattaagttaccacatcattatca

SEQ ID NO: 9 - DNA, 90 bp, Artificial Sequence
  Forward primer for gene Delta Sp-ccdB-amp Delta Sp-ccdB-F
actggtaagagatgttggtaacacctcttttactagtgactcttttgtgtgcactatgtaggcgatcgctttgtttatttttctaaatac

SEQ ID NO: 10 - DNA, 90 bp, Artificial Sequence
  Reverse primer for gene  Delta Sp-ccdB-amp Delta Sp-ccdB-R
agtacaagtttacaccaaagcaaaattaatactattaagaaggacgtgggactttggatcatgcgatcgctttgttcaaaaaaaagccag

(19) United States
(12) Patent Application Publication
(10) Pub. No.: US 2022/0204568 A1
(43) Pub. Date: Jun. 30, 2022

(54) METHOD FOR RAPID PREPARATION OF EPIDEMIC INFECTIOUS BRONCHITIS VACCINE

(71) Applicant: SOUTH CHINA AGRICULTURAL UNIVERSITY, Guangdong (CN)

(72) Inventors: Qingmei XIE, Guangdong (CN); Keyu FENG, Guangdong (CN); Jun FU, Guangdong (CN); Xinheng ZHANG, Guangdong (CN); Guanming Shao, Guangdong (CN)

(73) Assignee: SOUTH CHINA AGRICULTURAL UNIVERSITY, Guangdong (CN)

(21) Appl. No.: 17/696,646

(22) Filed: Mar. 16, 2022

(30) Foreign Application Priority Data
Nov. 22, 2019 (CN) 201911161596.2
Nov. 5, 2020 (CN) PCT/CN2020/126654

(51) Int. Cl. C07K 14/005; C12N 7/00

(52) U.S. Cl. CPC C07K 14/005; C12N 7/00; C12N 2770/20034; C12N 2770/20022; C12N 2770/20021

(57) ABSTRACT

The present disclosure provides a method for rapid preparation of an epidemic infectious bronchitis vaccine. An infectious clone of an infectious bronchitis virus (IBV) H120 vaccine strain is used as a skeleton carrier and an antigen gene in the skeleton carrier is replaced with a target antigen gene of an epidemic infectious bronchitis virus strain, to obtain a recombinant bronchitis virus. The target antigen gene is an S1 gene or an S gene. The S gene is one of the S gene fragments of the epidemic infectious bronchitis virus strain or a fusion gene composed of multiple S gene fragments. Furthermore, the target antigen gene and an N gene can be simultaneously replaced and a signal peptide region of the original S1 gene in the skeleton carrier is retained during the replacement.

The present application claims priority to International Application Number PCT/CN2020/126654 filed on Nov. 5, 2020 which claimed priority to the Chinese Patent Application No. CN201911161596.2, filed with the China National Intellectual Property Administration (CNIPA) on Nov. 22, 2019, and entitled “METHOD FOR RAPID PREPARATION OF EPIDEMIC INFECTIOUS BRONCHITIS VACCINE”, which are incorporated herein by reference in their entirety.

### TECHNICAL FIELD

The present disclosure relates to the technical field of biotechnology and particularly to a method for rapid preparation of an epidemic infectious bronchitis vaccine.

### BACKGROUND ART

Avian infectious bronchitis (IB) is an acute and highly contagious disease of chickens caused by an avian infectious bronchitis virus (IBV). It is also one of the poultry B/second-class infectious diseases prescribed by office international des épizooties (OIE) and China. IB is characterized by respiratory symptoms, nephritis and reduced egg-laying performance, particularly by a high mortality caused by infection of broiler chickens with renal infectious bronchitis characterized by kidney swelling. The mortality of white feather broilers reaches 30% or above and the mortality of high-quality broilers is about 15%. Weight gain and feed conversion rate of sick broilers are reduced. The IB has a mixed infection with Escherichia coli, mycoplasma and the like to cause airsacculitis to reduce quality of the broilers. In addition to causing death, infection of chicks with IB can also cause permanent irreversible damage to the reproductive system, which is manifested as dysplasia of oviducts and ovaries, or massive hydrocele and cysts in the oviducts and uterus, resulting in false hens, manifested as no peak of egg production, decreased egg production and quality, production of white-shelled, soft-shelled, sand-like-shelled and malformed eggs. Some IBV virus strains can also cause lesions in the intestines, proventriculus and muscles. IB can cause harm to chicken flocks of all ages and breeds and cause huge economic loss to the global poultry industry and is one of major infectious diseases which seriously affect the world poultry industry.

At present, the vaccine is a main measure to prevent and control avian IB and mainly divided into the following types on the market: 1) inactivated vaccines: the inactivated vaccines cannot be replicated in vivo and do not have a risk of pathological injury caused by returned strong virulence; they have a short induced immune response duration, only induce a humoral immune response, and do not induce a cellular immune response. Therefore, when the inactivated vaccines are used for immunization, an adjuvant needs to be matched and multiple vaccinations are needed, such that production cost of the vaccines is increased and popularization and application of the vaccines are limited. The inactivated vaccines must be inoculated by injection, which is difficult or even impossible to implement in a large-scale poultry breeding environment. Meanwhile, an adverse reaction at an injection site may lead to reduced carcass value; 2) attenuated live vaccines: they are time-consuming and labor-consuming during a preparation process, have a risk of in vivo mutation, recombination and returned strong virulence, and may be neutralized by maternal antibodies to reduce vaccine response; and 3) recombinant DNA vaccines: the recombinant DNA vaccine has a rigid preparation technology, and the post-translational protein modifications may alter protein immunogenicity, some of the recombinant DNA vaccines may need efficient carrier delivery.

However, since the 1990s, IB still continuously broken out in China, and has been widespread, thus bringing huge economic loss to the poultry breeding industry. The reason is that during a replication process of IBV in host cells, due to a lack of a proofreading function and low fidelity of RNA polymerase (RDRP), genome is prone to point mutation, insertion and deletion. Especially, the point mutation in S1 gene is likely to cause changes of neutralizing antigenic sites, resulting in generation of new serotypes and genotypes. Therefore, the difficulty in preventing and controlling IBV is that there are various serotypes and genotypes, but there is no or very weak cross-protection among different serotypes and genotypes.

### SUMMARY

The present disclosure overcomes problems that time and labor are needed to isolate and purify viruses, attenuation of continuous passage viruses is uncertain, returned virulence is strong during field application and the like in a traditional attenuated vaccine development process, avoids defects of high cost, long production period, possibility of polluted by exogenous viruses and the like, and provides a method for rapid preparation of a recombinant virus vaccine with strong timeliness and high rescue efficiency for epidemic infectious bronchitis.

In the method provided by the present disclosure, an infectious clone of an infectious bronchitis virus (IBV) H120 vaccine strain is used as a skeleton carrier, an antigen gene in the skeleton carrier is replaced with a target antigen gene of an epidemic infectious bronchitis virus strain, to obtain a recombinant bronchitis virus; the target antigen gene is an S gene or an S1 gene. The S gene is fused by S gene fragments of an epidemic infectious bronchitis virus strain with different serotypes/genotypes; and a signal peptide region of the original S1 gene in the skeleton carrier is retained during the replacement.

The S gene in the present disclosure can also be replaced with one of an S gene fragment of the infectious bronchitis epidemic virus strain or a fusion gene composed of multiple S gene fragments; and the serotypes or genotypes of the epidemic infectious bronchitis virus strain are different from those of an infectious bronchitis virus H120 vaccine strain.

In the present disclosure, the fusion gene refers to a hybrid gene formed by splicing gene sequences with different origins and functions through recombination and is also called a chimeric gene.

An S protein encoded by an IBV S gene is a highly glycosylated transmembrane protein located on virus surfaces, contains main antigen neutralizing epitopes of IBV, and can stimulate the body to produce specific neutralizing antibodies. After translation of the S gene, the resulting S protein is cut into two parts, an S1 protein and an S2 protein. The S1 protein locates at an amino terminal end and is one of the most important immunogenic components of the virus, contains the epitopes that induce production of neutralizing antibodies and binds a receptor through interaction between the viruses and cells. Moreover, the S1 protein is related to tissue affinity and virulence of the virus strain and its N-terminus determines a serotype difference of the IBV. A mutation of an S1 gene is closely related to an antigenic drift and pathogenic changes of the IBV.

In the present disclosure, a target antigen gene S gene is fused with S gene fragments from different serotypes and/or genotypes of an epidemic infectious bronchitis virus strain, such that a recombinant virus has a higher antibody titer. A new replacement method is also provided to provide more possibilities for multiple serotypes and/or genotype IB vaccines.

In addition, it is found that retaining an original signal peptide region of the S1 gene in a skeleton carrier is a key factor for successfully rescuing and obtaining the recombinant virus in a comparative experiment of two groups that retain or do not retain the original signal peptide region of the S1 gene in the skeleton carrier.

Furthermore, based on an example of the present disclosure, the fusion gene may be fused by the S1 gene of a serotype infectious bronchitis epidemic virus strain and an S2 gene of another serotype infectious bronchitis virus strain; or the fusion gene may be fused by the S1 gene of a genotype infectious bronchitis epidemic virus strain and an S2 gene of another genotype infectious bronchitis virus strain.

An S2 protein encoded by the S2 gene is very conserved in most IBV strains. In addition to anchoring the S1 protein, it also has the function of inducing cell-mediated immune responses and cross-reactive ELISA antibodies.

Furthermore, the S2 gene may be derived from the epidemic infectious bronchitis virus strain or an infectious bronchitis virus vaccine strain.

This study finds that the source of the S2 gene is not limited to the epidemic infectious bronchitis virus strain, use of a safer vaccine virus can also enable the fusion gene to have immunogenicity to infectious bronchitis viruses with a genotype and serotype of the vaccine virus strain, such that the body is stimulated to produce corresponding antibodies.

Further, the S1 gene may be an S1 fragment containing a hypervariable region.

An amino acid sequence of an S1 protein has a hypervariable region (HVR). The HVR constitutes serotype-specific and genotype-specific antigenic determinants of IBV and has a strong correlation with immune protection. Mutations in the HVR may affect changes of virus subgroups and cause appearance of new variants with different pathogenicity and virulence. Therefore, only when the replaced region contains the HVR, specific immune responses of body can be stimulated and IB neutralizing antibodies against this subtype are produced.

Furthermore, the method may include the following steps:

A1: conducting isolation to obtain two highly pathogenic epidemic infectious bronchitis wild virus strains of different serotypes or different genotypes, respectively extracting RNA and conducting RNA reverse transcription to obtain cDNA, and conducting amplification, by using each cDNA as a template, to correspondingly obtain an S2₁ gene and an S2₂ gene, and an S1₁ gene and an S1₂ gene that do not include the signal peptide region;

A2: fusing the S1₁ gene and S2₂ gene in step A1 or the S1₂ gene and S2₁ gene in step A1 to obtain a fusion gene S1₁+S2₂ or S1₂+S2₁;

A3: replacing the fusion gene S1₁+S2₂ or S1₂+S2₁ in step A2 on a constructed skeleton carrier of an H120 infectious clone by using a RED/ET technology and conducting screening to obtain a positive recombinant plasmid; and

A4: rescuing the positive recombinant plasmid in step A3 to obtain a recombinant virus that can immunize the two serotype or genotype infectious bronchitis viruses in step A1.

Furthermore, in step A3, the fusion gene S1₁+S2₂ is an S1 gene of GL15+ an S2 gene of GZ14, and the GenBank accession number of a GL15 sequence is KJ524616 and the GenBank accession number of a GZ14 sequence is KT946798.

Both GL15 and GZ14 are highly pathogenic and virulent strains of infectious bronchitis obtained in the early stage. The gene sequences have been published. In an embodiment, the S gene fused by the S1 gene of GL15+the S2 gene of GZ14 is used to replace the corresponding S1 and S2 genes in an H120 strain, but not limited to the two strains.

The present disclosure also provides another method for rapid preparation of an epidemic infectious bronchitis vaccine. An infectious clone of an infectious bronchitis virus H120 vaccine strain is used as a skeleton carrier and an antigen gene in the skeleton carrier is replaced with a target antigen gene of an epidemic infectious bronchitis virus strain to obtain a recombinant bronchitis virus. The difference is that the target antigen gene and an N gene are simultaneously replaced, and the target antigen gene is an S gene or an S1 gene. A signal peptide region of the original S1 gene in the skeleton carrier is retained during the replacement.

There are some T cell epitopes at a carboxyl end of an N protein encoded by a N gene lock which can stimulate cytotoxic T lymphocyte responses. Studies have found that an amino end of the N protein contains many linear B cell epitopes. A large number of antigenic determinants are distributed on the N protein which have very strong immunogenicity and can induce the body to produce antibodies.

The target antigen S gene or S1 gene is replaced with the N gene at the same time which avoids the pre-existing immune interference of the N gene in the original skeleton carrier to the target antigen S gene or S1 gene and strengthens specific immune responses.

Furthermore, as a preferred embodiment of the present disclosure, the S1 gene may be simultaneously replaced with the N gene.

Furthermore, the infectious bronchitis epidemic virus strain may be a highly pathogenic wild virus strain.

Furthermore, the S1 gene and N gene may be derived from GL15 and the GenBank accession number of a GL15 sequence is KJ524616.

The highly pathogenic strain has better immunogenicity and can better stimulate specific immune responses of recombinant vaccines in the body.

Compared with the prior art, the present disclosure has the following of advantages and effects:

The method for rapid preparation of an epidemic infectious bronchitis vaccine is simple and easy to operate, has high repeatability and good generation stability, and can rapidly and efficiently deal with frequently mutated epidemic IB. Besides, the method provides a new idea for construction of a vector vaccine.

The recombinant vaccine produced by the present method was safe and reliable and can be used for all ages without mutual interference. The recombinant vaccine produced by the present disclosure can be taken orally or intranasally, can meet requirements of large-scale application and low cost, has a high protective effect, high homology protection and small cilia damage, and enables fallopian tube to develop normally. Besides, the recombinant IBV is live virus and has similar ways of infection, replication and immune response to those of traditional IBV vaccines.

### BRIEF DESCRIPTION OF THE DRAWINGS

FIG. 1 is a RT-PCR detection result diagram of a recombinant virus rH120-ΔS1z-ΔNz/GL15, where lanes 1 and 2: S1 and N genes of an H120 strain; lanes 3 and 4: S1 and N genes of a rH120-ΔS1z-ΔNz/GL15 strain; N: Blank control; and M: DL2,000 DNA Marker;

FIG. 2 is a Western-blot diagram for identifying a recombinant virus rH120-ΔS1z-ΔNz/GL15;

FIG. 3 is a growth curve determination graph of a recombinant virus rH120-ΔS1z-ΔNz/GL15;

FIG. 4 is a RT-PCR detection result diagram of a recombinant virus rH120-ΔSp/GL15-GZ14, where lanes 1 and 2: S and N genes of an H120 strain; lanes 3 and 4: S and N genes of a rH120-ΔSp/GL15-GZ14 strain; N: Blank control; and M: DL5,000 DNA Marker;

FIG. 5 is a Western-blot diagram for identifying a recombinant virus rH120-ΔSp/GL15-GZ14; and

FIG. 6 is a growth curve determination graph of a recombinant virus rH120-ΔSp/GL15-GZ14.

### DETAILED DESCRIPTION OF THE EMBODIMENTS

The present disclosure is further illustrated with reference to the specific examples, which are not intended to limit the present disclosure in any form. Unless otherwise specified, the reagents, methods and equipment used in the present disclosure are conventional in the art.

Virus strain: vaccine strain H120 strain (Accession number: FJ807652). QX type IBV virulent strains GL15 and GZ14 are isolated and preserved by our laboratory, their sequences have been published and the GenBank accession number are KJ524616 and KT946798, respectively. A recombinant poxvirus MVA-T7 expressing a T7 RNA polymerase is kindly donated by Dr. Bernard Moss of the National Institutes of Health (NIH).

Cells: BHK Cells and CK Cells

Plasmids and strains: an H120 infectious clone pBR322-H120 and a helper plasmid pVAX1-H120 N are constructed by researchers of this study; a eukaryotic expression vector pVAX1 is a product of Invitrogen; an eukaryotic expression vector pEGFP-C1 is a product of Clontech; and eukaryotic expression plasmids pRK5-GL15 S, pRK5-GL15 N and pRK5-GZ14 S are constructed and preserved by our laboratory. Escherichia coli DH5a competence is a product of Shanghai Weidi Biotechnology Co., Ltd. A plasmid pBR322-amp-ccdB-rpsLneo and Escherichia coli DH10B and DH10B gyrA462 expressing a recombinant protein Redα/Redβ are kindly donated by Professor Zhang Youming of Shandong University.

Experimental chicken embryos and animals: SPF chicken embryos (9-11 days old) and 2-day-old SPF chickens are purchased from the SPF Experimental Animal Center of Guangdong Xinxing Dahua Agricultural Poultry Egg Co., Ltd.

Tool enzymes and main reagents: DNA polymerase (PrimeSTAR Max DNA Polymerase), one-step RT-PCR kit (PrimeScript One Step RT-PCR Kit Ver.2), Recombinant DNase I (RNase-free), DNA Marker DL2000, DNA Marker DL15000, 6× Loading Buffer which are products of Takara Biotechnology (Dalian) Co., Ltd., Restriction enzymes AsiSI, PacI, BamH I, Xhol, PmeI, Bstzl7I, DpnI and 1 Kb DNA Ladder are products of New England Biolabs (NEB). An Axyprep Body Fluid Viral DNA/RNA Miniprep kit is a product of Axygen. a Gel Extraction Kit, a PCR product purification kit (Cycle Pure Kit), a plasmid mini extraction kit (Plasmid Mini Kit), an endotoxin-free plasmid maxi extraction kit (Endo-Free Plasmid Maxi Kit), plasmid extraction reagents (Solution I, II and III) and RNase A are purchased from OMEGA, agarose is purchased from Biowest; a nucleic acid dye Gelred is a product of Sangon Biotech (Shanghai) Co., Ltd., and other chemical reagents such as absolute ethanol, isopropanol, phenol, chloroform, sodium acetate, etc. are domestically analytically pure products.

Unless otherwise specified, the reagents and materials used in the present disclosure are commercially available.

### EXAMPLE 1

In this example, the experiment was performed in a replacement mode of S1+N.

1) PCR Amplification of Screening Marker Genes with Homologous Arms of Target Replacement Genes (S1 and N Genes on GL15)

A plasmid pUC57-amp-ccdB was used as a template and primers ΔS1z-ccdB-F/R and ΔNz-ccdB-F/R are used to conduct a PCR amplification of screening marker genes ΔS1z/ccdB-amp and ΔNz/ccdB-amp with homologous arms.

primer ΔS1z-ccdB-F:
ACTGGTGAAGAGTGTTGGTGACACCTCTTTTGCTGGTGGCTCTTTTGTGT
GCACTGTGTGGGCGATCGCTTTGTTTGTTTTTCTGAATGC, as set
forth in SEQ ID NO: 1;
primer ΔS1z-ccdB-F-R:
ATGTGTCTGAAATGGCCAAACCTGCGTCTGCTGAGTGATTGTGACTGACA
GCTGGGTCGGTGCGATCGCTTTGTTCAAAAAAAAGCCAG, as set
forth in SEQ ID NO: 2;
primer ΔNz-ccdB-F:
CGGTAAAGCAACTGGAGAAGCAAGCGCCCCAGCGCCAGTCATCAAACTAG
GCGATCGCTTTGTTTATTTTTCTAAAT, as set forth in SEQ
ID NO: 3;
and
primer ΔNz-ccdB-R:
AGGAGAAAACAGCATACGCACGTCCATAGTGTTAAGTTGAGAATCTAGCT
GCGATCGCTTTGTTCAAAAAAAAGC, as set forth in SEQ ID
NO: 4.

A PCR amplification system was shown in Table 1. 5 μL of a PCR product was subjected to 1% agarose gel electrophoresis. An amplification result was observed. The PCR product was observed to be about 1.5 Kb, which was consistent with expectations. The remaining PCR product was recovered and purified by a PCR product purification kit.

TABLE 1
PrimeSTAR Max DNA polymerase PCR reaction system

| Reaction Component | 50-μL Reaction System |
|---|---|
| 2x PrimeSTAR Max DNA Polymerase | 25 μL |
| Template DNA | 1 μL |
| Forward primer, 10 μM | 2 μL |
| Reverse primer, 10 μM | 2 μL |
| ddH₂O | supplementing to 50 μL |

The recovered product was digested by restriction enzymes DpnI and subjected to 1% agarose gel electrophoresis. The digested product was recovered and purified by using a gel extraction kit, the nucleic acid concentration was determined, and a small amount of the recovered product was sent to BGI for sequencing.

Recombination of Screening Marker Gene with pBR322-H120:

100 ng each of the screening marker gene ΔS1z/ccdB-amp and an infectious clone pBR322-H120 was added to a newly prepared DH10B gyrA462 electroporated competence that induced an expression of a Redα/Redβ recombinant protein. A tube bottom of a 1.5-mL centrifuge tube was flicked with hands and gently evenly mixed. The mixture was transferred to a 1-mm electroporation cup and the electroporation cup was placed in an electroporation device. Electroporation was conducted according to a set RED/ET electroporation program. After the electroporation was completed, 1 mL of an SOC medium was added immediately to the electroporation cup, bacteria were resuspended and transferred to a 2-mL centrifuge tube.

The electroporated bacterial solution was placed on a 37° C. shaker, shaken at 260 rpm and cultivated for 60 min to complete recombination and resistance recovery. The bacterial solution was centrifuged and precipitated to obtain bacteria and most of the supernatant was discarded. The bacteria were pipetted to resuspend and spread all on an LB plate containing chloramphenicol and ampicillin. The plate was placed in an incubator for culture at 37° C. for 16 h.

Identification of Recombinant Plasmid Containing Screening Marker Gene:

Colonies were picked from the plate and inoculated in a 5-mL LB liquid medium containing chloramphenicol and ampicillin, the bacteria were cultured at 37° C. and at 260 rpm in a shaking manner for 16 h, 3 mL of a bacterial solution was taken for extracting plasmids by using a plasmid small extraction kit, then a restriction endonuclease identification was conducted (a restriction enzyme digestion system was seen in Table 2), reaction was conducted at 37° C. for 2 hours, a digested product was subjected to 1% agarose gel electrophoresis, a result was observed, the identified correct plasmids were be sent to BGI for sequencing and the recombinant plasmids were named pBR322-H120-ΔS1z/ccdB-amp.

TABLE 2
Enzyme digestion system

| Reaction Component | 10-μL Reaction System | 50-μL Reaction System |
|---|---|---|
| Restriction enzyme | 0.2 μL | 1 |
| 10 × Cutsmart buffer | 1 μL | 5 |
| DNA | 1 μg | 4 μg |
| ddH₂O | supplementing to 10 μL | supplementing to 50 μL |

Functional Verification of Screening Marker Gene:

The prepared recombinant plasmids pBR322-H120-ΔS1z/ccdB-amp were electroporated into DH10B gyrA462 and DH10B electroporated competence, respectively. After transformation and recovery, the bacteria solution was spread on an LB plate containing ampicillin, the plate was placed at 37° C. for overnight culture and growth of Escherichia coli was observed.

A result was shown in the figure. Transformants that can grow normally in DH10B gyrA462 are successfully recombined.

Preparation of S1 and N Replacement Genes:

Plasmids pRK5-GL15 S and pRK5-GL15 N were respectively as templates, primers ΔS1z-GL15-F/R and ΔNz-GL15-F/R were used to perform PCR amplification of S1 and N replacement genes ΔS1z/GL15 and ΔNz/GL15 containing homologous arms. PCR amplification was conducted by using high-fidelity DNA polymerase PrimeSTAR Max DNA Polymerase (Takara).

TABLE 3
Amplification system

| Reaction Component | 50-μL Reaction System |
|---|---|
| 2x PrimeSTAR Max DNA Polymerase | 25 μL |
| Template DNA | 1 μL |
| Forward primer, 10 μM | 2 μL |
| Reverse primer, 10 μM | 2 μL |
| ddH₂O | supplementing to 50 μL |

PCR amplification parameters were as follows: 98° C. for 2 min; 98° C. for 10 s, 55° C. for 5 s, 72° C. for 30 s, 30 cycles of amplification, and 72° C. for 2 min.

PCR product was subjected to 1% agarose gel electrophoresis. An amplification result was observed. A gel extraction kit was used for recovery and purification. The nucleic acid concentration was determined. A small amount of the recovered and purified product was sent to BGI for sequencing. In the sequence comparison result, the correct target genes were the same as the S1 and N in the original plasmid.

Screening Marker Gene Replaced by S1 Gene:

100 ng each of the replacement gene ΔS1z/GL15 and the recombinant plasmid pBR322-H120-ΔS1z/ccdB-amp containing the screening marker gene was added to a newly prepared DH10B electroporated competence, and electroporation and recovery were conducted according to the method and procedure in step 3). The bacterial solution was centrifuged and precipitated to obtain bacteria and most of the supernatant was discarded. The bacteria were pipetted to resuspend and spread all on an LB plate containing chloramphenicol. The plate was placed in an incubator for culture at 37° C. for 16 h.

Screening of Recombinant Plasmid:

Colonies were picked from the plate, the bacterial solution was subjected to enlarged culture under shaking, plasmids were extracted and restriction digestion identification was conducted. 5 mL of the correctly identified bacterial solution was added to 200 mL of an LB liquid medium containing chloramphenicol for enlarged culture. After shaking at 37° C. and 260 rpm for 16 h, the plasmids were extracted according to instructions of an endotoxin-free plasmid maxi extraction kit and the plasmids were freeze-stored at −20° C. A small amount of the plasmids were sent to BGI company for sequencing verification. The recombinant plasmids with correct sequencing were named pBR322-H120-ΔS1z/GL15.

Replacement of N Gene in Recombinant Plasmids pBR322-H120-ΔS1z/GL15:

On the basis of pBR322-H120-ΔS1z/GL15, 100 ng each of the screening marker gene ΔNz/ccdB-amp and pBR322-H120-ΔS1z/GL15 were added to the newly prepared DH10B gyrA462 electroporated competence that induced an expression of a Redα/Redβ recombinant protein, such as in step 3-5), the positive plasmids pBR322-H120-ΔS1z/GL15-ΔNz/ccdB-amp were obtained by recombination screening, in the second step of recombination, ΔNz/GL15 was replaced with ΔNz/ccdB-amp, and the correct recombinant plasmid was screened and named pBR322-H120-ΔS1z-ΔNz/GL15.

Rescue of Recombinant Plasmid:

DF-1 cells were passaged to a six-well plate the day before transfection. When the cells grew to about 70%, MVA-T7 poxvirus was inoculated into the cells according to MOI=1, the inoculated cells were placed in a 5% CO₂ incubator at 37° C. for 4 h, a medium was changed, the cells were washed twice with a PBS solution, and an antibiotics-free DMEM medium containing 2% serum was added.

The reaction system was prepared according to the instructions of a transfection reagent (jetPRIME transfection reagent (Polyplus)). Each well of a transfection mixture contained 2 μg of recombinant plasmids and 0.5 g of pVAX1-H120 N (total amount of DNA was 2.5 μg). The transfection mixture was added to cell wells, the transfected cells were cultured in a 5% CO₂ incubator at 37° C. for 4 h, the medium was changed and the cells continued to be cultured in a 5% CO₂ incubator at 37° C. During the transfection process, repeated tests, and a blank control group and a control test of a pBR322-cm empty vector were set up to eliminate test operation errors and virus contamination.

After the cells were cultured for 72 h, a culture dish was frozen at −80° C., the cells were thawed at room temperature, freezed-thawed for three times to break cells and centrifuged at 10,000×g for 5 min. A cell supernatant was filtered through a 0.22-μM filter membrane to remove the possible residual MVA-T7 poxvirus and then named FO. The cell supernatant was inoculated into five 9-day-old SPF chicken embryos through an allantoic cavity with 0.2 ml each embryo and incubated at 37° C. The chicken embryos were observed 24 h after the inoculation, the dead chicken embryos were discarded, and the cells were continuously incubated until 48 h to harvest allantoic fluid of the chicken embryos. In this way, the 5 generations were blindly passed and the allantoic fluid of the 5th generation chicken embryos was harvested and named F5. A recombinant virus rH120-ΔS1z-ΔNz/GL15 was obtained after filtration through a 0.22-μM filter membrane and finally sub-packaged and stored at −80° C.

11) Identification of Rescue Virus

RT-PCR Detection

200 μL allantoic fluid of the rH120-ΔS1z-ΔNz/GL15 virus was taken. Viral RNA was extracted according to the instructions of an Axyprep Body Fluid Viral DNA/RNA Miniprep kit. The extracted RNA was dissolved in 40 μL of RNase-free TE buffer. 10 μL of RNA was taken and DNA was removed according to instructions of Recombinant DNase I (RNase-free) (Takara). The above RNA was used as a template, primers IBV-S1-F/R and IBV-N-F/R were used and the S1 gene and N gene were amplified according to the instructions of one-step RT-PCR kit (PrimeScript One Step RT-PCR Kit Ver.2). PCR amplification program was as follows: 50° C. for 30 min; 95° C. for 4 min; 95° C. for 30 sec, 53° C. for 30 sec, 72° C. for 1 min and 40 sec, and a total of 35 cycles; and extension at 72° C. for 5 min and storage at 4° C.

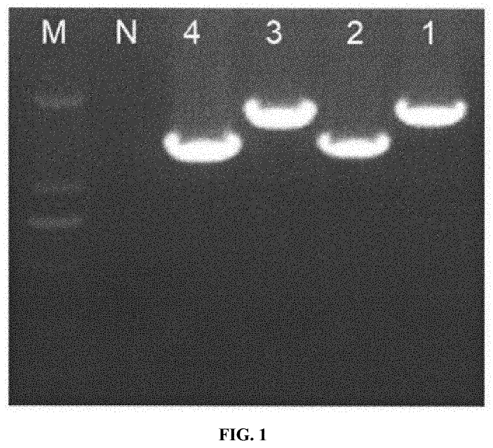

The RT-PCR product was subjected to 1% agarose gel electrophoresis and observed (see FIG. 1). Bands were recovered and sent to BGI company for sequencing. The sequencing result showed that the product was 100% homologous to S1 and N nucleotide sequences of the target antigen gene, indicating that the target antigen S1 and N genes had successfully replaced the S1 and N on the original H120 strain.

Western-Blot Identification

The rH120-ΔS1z-ΔNz/GL15 virus was inoculated into a monolayer of CK cells (MOI=1) in a cell culture six-well plate, after conducting adsorption at 37° C. for 2 h, exchange DMEM medium containing 2% serum, the cells were placed at 5% CO₂ incubator for culture at 37° C. for 48 h, cell proteins were harvested, the medium in each well of the cell culture plate was aspirated, the cell proteins were washed three times with PBS, a cell lysate containing 1% of a protease inhibitor was added, and boiling was conducted for 5 min. SDS-PAGD electrophoresis was conducted after a separation gel and a concentrated gel were prepared. 35 μg of each sample protein solution was taken, ¼ volume of 5× loading buffer was added, and the samples were boiled on a heater set at 100° C. for 5 min. A vertical electrophoresis tank was connected to an electrophoresis instrument, the voltage of which was set to 80 V. When bromophenol blue dye entered the separation gel, the voltage increased to 160 V. After the electrophoresis was completed, the gel was taken for subsequent Western blotting detection.

Transfer from the completed SDS-PAGE gel to a PVDF membrane was conducted. A wet transfer method was used in this study. A transfer solution was pre-cooled in a refrigerator at 4° C. The SDS-PAGE gel was transferred to the transfer solution for soaking. The PVDF membrane was tailored according to a size of a gel of a targeted protein. Since an M protein had a smaller molecular weight, the PVDF membrane with a pore size of 0.45 μm was used. A transfer condition was membrane transfer at constant current of 200 mA for 1 h; and after the transfer, the PVDF membrane was taken out and sealed with 5% skimmed milk powder at room temperature for 1 h (or overnight at 4° C.). A mouse-derived IBV M protein monoclonal antibody was diluted with TBST at a ratio of 1:1,000 and incubated at room temperature for 1 h; and the membrane was washed with the TBST at room temperature for 3 times, each time for 10 min. FITC-labeled goat anti-mouse IgG was diluted at a ratio of 1:10,000 and incubated at room temperature for 1 h; and the secondary antibody was discarded and the membrane was washed with the TBST for 3 times, each time for 5 min. Liquid A and liquid B in an ultra-sensitive ECL chemiluminescence kit were mixed at a ratio of 1:1, filter paper was used to absorb the remaining TBST on the PVDF membrane, the membrane was put on an Azure C600 imaging system workbench, luminescent liquid was added dropwise on the membrane, and parameters of the instrument were adjusted for exposure.

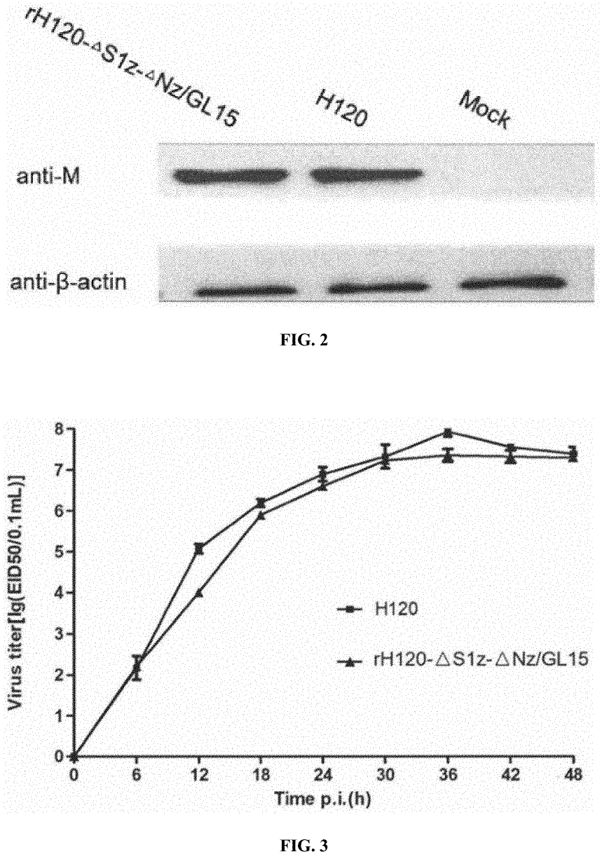

A result was shown in FIG. 2. Both the rH120-ΔS1z-ΔNz/GL15 recombinant virus and the vaccine virus strain H120 can express the M protein, which proved that the rH120-ΔS1z-ΔNz/GL15 recombinant virus was successfully rescued.

Determination of Growth Curve of rH120 Virus

The rH120-ΔS1z-ΔNz/GL15 virus solution and the mother virus H120 were diluted with normal saline, and inoculated into thirty 10-day-old SPF chicken embryos through an allantoic cavity with 100 EID50 (50% egg infections dose)/embryo. Virus allantoic fluids of five chicken embryos were harvested at 6 h, 12 h, 24 h, 36 h and 48 h respectively after the inoculation, mixed, sub-packaged and freeze-stored at −80° C. A Reed-Muench method was used to determine the EID50 of the virus at different time points and a growth curve was plotted.

A result was shown in FIG. 3. The rH120-ΔS1z-ΔNz/GL15 recombinant virus had a growth curve similar to that of the original vaccine strain H120 and can proliferate normally.

Immune effect experiment of recombinant virus rH120-ΔS1z-ΔNz/GL15 as a vaccine against GL15 virus challenge.

An experimental group was a 2-day-old immune recombinant virus rH120-ΔS1z-ΔNz/GL15 group.

A control group 1 was a 2-day-old immune virus vaccine H120 group.

A control group 2 was a 2-day-old immune-free group.

A blank group was an immune-free and challenge-free group.

TABLE 4
Morbidity and mortality of each group after challenge

| Group | Morbidity (%) | Mortality (%) |
|---|---|---|
| Experimental group | 0%^{a} | 0%^{a} |
| Control group 1 | 70%^{b} | 50%^{b} |
| Control group 2 | 100%^{b} | 70%^{b} |
| Blank group | 0%^{a} | 0%^{a} |

It can be seen that the recombinant virus had a good immune effect and can provide 100% protection against GL15.

### EXAMPLE 2

This example was a replacement of a fusion GL15-GZ14 S gene.

1) Preparation of targeted genes GL15-S1 and GZ14-S2: plasmids pRK5-GL15 S and pRK5-GZ14 S were respectively used as templates, and primers GL15-S1-F/R and GZ14-S2-F/R were used to amplify GL15-S1 and GZ14-S2 containing homologous arms, respectively. PCR amplification was conducted by using high-fidelity DNA polymerase PrimeSTAR Max DNA Polymerase (Takara). Referring to the preparation system in Table 1, PCR amplification parameters were: 98° C. for 2 min; 98° C. for 10 s; 55° C. for 5 s; 72° C. for 20 s; and amplification for 30 cycles and extension at 72° C. for 5 min. PCR product was subjected to 1% agarose gel electrophoresis. A gel extraction kit was used for recovery and purification. The nucleic acid concentration was determined. A small amount of the recovered and purified product was sent to BGI for sequencing.

primer GL15-S1-F:
ACTGGTAAGAGATGTTGGTAACACCTCTTTTACTAGTGACTCTTTTGTGT
GCACTATGTGCAAATTTGTTAGATCTTAC, as set forth in SEQ
ID NO: 5;
primer GL15-S1-R:
ACAACTAGTAACATTACTACTAATAGAACGTCTAAAACGACGTGAGCTAT
TGGTTAACTTAAC, as set forth in SEQ ID NO: 6;
primer GZ14-S2-F:
ACAGGTTGAGAACCAGTTTTATGTTAAGTTAACCAATAGCTCACGTCGTT
TTAGACGTTCTATTAG, as set forth in SEQ ID NO: 7;
and
primer GZ14-S2-R:
ACAAGTTTACACCAAAGCAAAATTAATACTATTAAGAAGGACGTGGGACT
TTGGATCATTAAGTTACCACATCATTATCA, as set forth in
SEQ ID NO: 8.

2) Preparation of Screening Marker Genes

A plasmid pUC57-amp-ccdB was used as a template and primers ΔSp-ccdB-F/R were used to conduct a PCR amplification of screening marker genes ΔSp/ccdB-amp with homologous arms. A PCR amplification system was shown in Table 1. 5 μL of a PCR product was subjected to 1% agarose gel electrophoresis. An amplification result was observed. The remaining PCR product was recovered and purified by a PCR product purification kit. The recovered product was digested by restriction enzymes DpnI and subjected to 1% agarose gel electrophoresis. The digested product was recovered and purified by using a gel extraction kit, the nucleic acid concentration was determined, and a small amount of the recovered product was sent to BGI for sequencing.

ΔSp-ccdB-F:
ACTGGTAAGAGATGTTGGTAACACCTCTTTTACTAGTGACTCTTTTGTGT
GCACTATGTAGGCGATCGCTTTGTTTATTTTTCTAAATAC, as set
forth in SEQ ID NO: 9;
ΔSp-ccdB-R:
AGTACAAGTTTACACCAAAGCAAAATTAATACTATTAAGAAGGACGTGGG
ACTTTGGATCATGCGATCGCTTTGTTCAAAAAAAAGCCAG, as set
forth in SEQ ID NO: 10.

3) Recombination of Screening Marker Gene with pBR322-H120

100 ng each of the screening marker gene Sp/ccdB-amp and an infectious clone pBR322-H120 was added to a newly prepared DH10B gyrA462 electroporated competence that induced an expression of a Redα/Redβ recombinant protein, and gently evenly mixed. The mixture was transferred to a 1-mm electroporation cup and the electroporation cup was placed in an electroporation device. Electroporation was conducted according to a set RED/ET electroporation program. After the electroporation was completed, 1 mL of an SOC medium was added immediately to the electroporation cup, bacteria were resuspended and transferred to a 2-mL centrifuge tube. The electroporated bacterial solution was placed on a 37° C. shaker, shaken at 260 rpm and cultivated for 60 min to complete recombination and resistance recovery. The bacterial solution was centrifuged and precipitated to obtain bacteria and most of the supernatant was discarded. The bacteria were pipetted to resuspend and spread all on an LB plate containing chloramphenicol and ampicillin. The plate was placed in an incubator for culture at 37° C. for 16 h. The positive clone was picked as pBR322-H120-ΔSp/ccdB-amp.

4) Screening Marker Gene Replaced by Fusion of GL15-S1 and GZ14-S2 Genes

100 ng each of GL15-S1, GZ14-S2 and pBR322-H120-ΔSp/ccdB-amp containing homologous arms was added into a newly prepared DH10B electroporated competence, and electroporation and recovery were conducted according to the method and procedure in step 3). The bacterial solution was centrifuged and precipitated to obtain bacteria and most of the supernatant was discarded. The bacteria were resuspended and spread all on an LB plate containing chloramphenicol. The plate was placed in an incubator for culture at 37° C. for 16 h.

5) Screening of Recombinant Plasmid

Colonies were picked from the plate, the bacterial solution was subjected to enlarged culture under shaking according to the method in Example 1, plasmids were extracted and restriction digestion identification was conducted. The restriction digestion identification was shown in FIG. 2.5 mL of the correctly identified bacterial solution was added to 200 mL of an LB liquid medium containing chloramphenicol for enlarged culture. After shaking at 37° C. and 260 rpm for 16 h, the plasmids were extracted according to instructions of an endotoxin-free plasmid maxi extraction kit and the plasmids were freeze-stored at −20° C. A small amount of the plasmids were sent to BGI company for sequencing verification. The recombinant plasmids with correct sequencing were named pBR322-H120-ΔSp/GL15-GZ14.

6) The recombinant plasmid rescue method was the same as in Example 1 to obtain a recombinant virus rH120-ΔSp/GL15-GZ14.

7) Identification of Rescue Virus

RT-PCR Detection

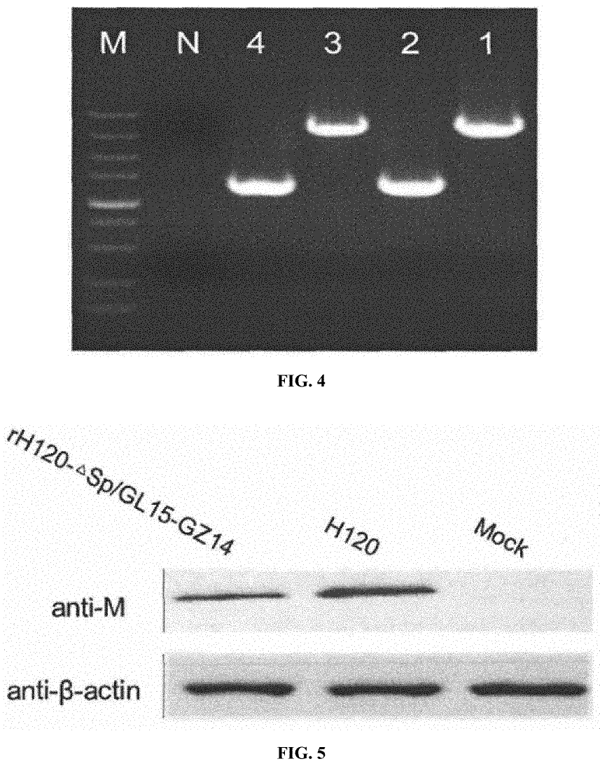

200 μL allantoic fluid of the rH120-ΔSp/GL15-GZ14 virus was taken. Viral RNA was extracted according to the instructions of an Axyprep Body Fluid Viral DNA/RNA Miniprep kit. The extracted RNA was dissolved in 40 μL of RNase-free TE buffer. 10 μL of RNA was taken and DNA was removed according to instructions of Recombinant DNase I (RNase-free) (Takara). The above RNA was used as a template, primers IBV-S-F/R and IBV-N-F/R were used and an S gene and an N gene were amplified according to the instructions of one-step RT-PCR kit (PrimeScript One Step RT-PCR Kit Ver.2). PCR amplification program was as follows: 50° C. for 30 min; 95° C. for 4 min; 95° C. for 30 sec, 53° C. for 30 sec, 72° C. for 2 min, and a total of 35 cycles; and extension at 72° C. for 5 min and storage at 4° C. The RT-PCR product was subjected to 1% agarose gel electrophoresis and observed (see FIG. 4), indicating that the target antigen S1 and S2 genes had successfully replaced S1 and S2 on the original H120 virus strain. Bands were recovered and sent to BGI company for sequencing. A sequencing result showed that the RT-PCR product was 100% homologous to the S1 nucleotide sequence of the target antigen gene GL15 and the S2 nucleotide sequence of the target antigen gene GZ14.

Western-Blot Identification

The rH120-ΔSp/GL15-GZ14 virus solution was inoculated into a monolayer of CK cells (MOI=1) in a cell culture six-well plate, after conducting adsorption at 37° C. for 2 h, the solution was inoculated in to a DMEM medium containing 2% serum, the cells were placed at 5% CO₂ incubator for culture at 37° C. for 48 h, cell proteins were harvested, the medium in each well of the cell culture plate was aspirated, the cell proteins were washed three times with PBS, a cell lysate containing 1% of a protease inhibitor was added, and boiling was conducted for 5 min. SDS-PAGD electrophoresis was conducted after a separation gel and a concentrated gel were prepared. 35 μg of each sample protein solution was taken, ¼ volume of 5× loading buffer was added, and the samples were boiled on a heater set at 100° C. for 5 min. A vertical electrophoresis tank was connected to an electrophoresis instrument, the voltage of which was set to 80 V. When bromophenol blue dye entered the separation gel, the voltage increased to 160 V. After the electrophoresis was completed, the gel was taken for subsequent Western blotting detection.

Transfer from the completed SDS-PAGE gel to a PVDF membrane was conducted. A wet transfer method was used in this study. A transfer solution was pre-cooled in a refrigerator at 4° C. The SDS-PAGE gel was transferred to the transfer solution for soaking. The PVDF membrane was tailored according to a size of a gel of a targeted protein. Since an M protein had a smaller molecular weight, the PVDF membrane with a pore size of 0.45 μm was used. A transfer condition was membrane transfer at constant current of 200 mA for 1 h; and after the transfer, the PVDF membrane was taken out and sealed with 5% skimmed milk powder at room temperature for 1 h (or overnight at 4° C.). A mouse-derived IBV M protein monoclonal antibody was diluted with TBST at a ratio of 1:1,000 and incubated at room temperature for 1 h; and the membrane was washed with the TBST at room temperature for 3 times, each time for 10 min. FITC-labeled goat anti-mouse IgG was diluted at a ratio of 1:10,000 and incubated at room temperature for 1 h; and the secondary antibody was discarded and the membrane was washed with the TBST for 3 times, each time for 5 min. Liquid A and liquid B in an ultra-sensitive ECL chemiluminescence kit were mixed at a ratio of 1:1, filter paper was used to absorb the remaining TBST on the PVDF membrane, the membrane was put on an Azure C600 imaging system workbench, luminescent liquid was added dropwise on the membrane, and parameters of the instrument were adjusted for exposure.

A result was shown in FIG. 5. Both the rH120-ΔSp/GL15-GZ14 recombinant virus and the vaccine virus strain H120 can express the M protein, which proved that the rH120-ΔSp/GL15-GZ14 recombinant virus was successfully rescued.

Determination of Growth Curve of rH120 Virus

The rH120-ΔSp/GL15-GZ14 virus solution and the mother virus H120 were diluted with normal saline, and inoculated into thirty 10-day-old SPF chicken embryos through an allantoic cavity with 100 EID50 (50% egg infections dose)/embryo. Virus allantoic fluids of five chicken embryos were harvested at 6 h, 12 h, 24 h, 36 h and 48 h respectively after the inoculation, mixed, sub-packaged and freeze-stored at −80° C. A Reed-Muench method was used to determine the EID50 of the virus at different time points and a growth curve was plotted.

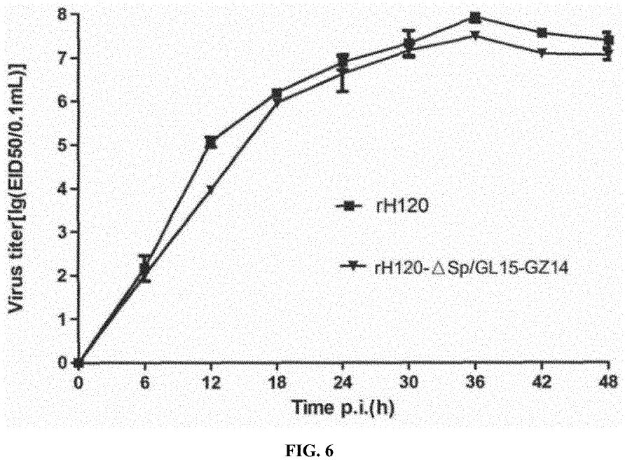

A result was shown in FIG. 6. The rH120-ΔSp/GL15-GZ14 recombinant virus had a growth curve similar to that of the original vaccine strain H120 and can proliferate normally.

7) Immune effect experiment of recombinant virus rH120-ΔSp/GL15-GZ14 as a vaccine against GL15 virus (QX type) and GZ14 virus (TW I type) challenge.

An experimental group 1 was a 2-day-old immune recombinant virus rH120-ΔSp/GL15-GZ14 and 16-day-old GL15 strain challenge group.

An experimental group 2 was a 2-day-old immune recombinant virus rH120-ΔSp/GL15-GZ14 and 16-day-old GZ14 strain challenge group.

A control group 1 was a 2-day-old immune virus vaccine H120 and 16-day-old GL15 strain challenge group.

A control group 2 was a 2-day-old immune virus vaccine H120 and 16-day-old GZ14 strain challenge group.

A control group 3 was a 2-day-old immune-free and 16-day-old GL15 strain challenge group.

A control group 4 was a 2-day-old immune-free and 16-day-old GZ14 strain challenge group.

A blank group was an immune-free and challenge-free group.

TABLE 5
Morbidity and mortality of each group after challenge

| Group | Morbidity (%) | Mortality (%) |
|---|---|---|
| Experimental group 1 | 0% | 0% |
| Experimental group 2 | 0% | 0% |
| Control group 1 | 80% | 40% |
| Control group 2 | 100% | 30% |
| Control group 3 | 100% | 30% |
| Control group 4 | 100% | 30% |
| Blank group | 0% | 0% |

It can be seen that the recombinant virus had a good immune effect, can 100% prevent the infection of the QX type and TW I type infectious bronchitis viruses, and significantly reduced the morbidity and mortality of infectious bronchitis caused by the QX type and TW I type infectious bronchitis viruses.

What is claimed is:

1. A method for rapid preparation of an epidemic infectious bronchitis vaccine, wherein an infectious clone of an infectious bronchitis virus (IBV) H120 vaccine strain is used as a skeleton carrier and an antigen gene in the skeleton carrier is replaced with a target antigen gene of an epidemic infectious bronchitis virus strain, to obtain a recombinant bronchitis virus; the target antigen gene is an S gene or an S1 gene; the S gene is fused by S gene fragments of an epidemic infectious bronchitis virus strain with different serotypes/genotypes; and a signal peptide region of the original S1 gene in the skeleton carrier is retained during the replacement.

2. The method for rapid preparation of an epidemic infectious bronchitis vaccine according to claim 1, wherein the S gene is replaced with one of S gene fragments of the epidemic infectious bronchitis virus strain or a fusion gene composed of multiple of the S gene fragments; and
the serotypes or genotypes of the epidemic infectious bronchitis virus strain are different from that of an infectious bronchitis virus H120 vaccine strain.

3. The method for rapid preparation of an epidemic infectious bronchitis vaccine according to claim 2, wherein the fusion gene is fused by the S1 gene of a serotype epidemic infectious bronchitis virus strain and an S2 gene of another serotype infectious bronchitis virus strain;
or the fusion gene is fused by the S1 gene of a genotype epidemic infectious bronchitis virus strain and an S2 gene of another genotype infectious bronchitis virus strain.

4. The method for rapid preparation of an epidemic infectious bronchitis vaccine according to claim 3, wherein the S2 gene is derived from the epidemic infectious bronchitis virus strain or an infectious bronchitis vaccine virus strain.

5. The method for rapid preparation of an epidemic infectious bronchitis vaccine according to claim 1, wherein the S1 gene is an S1 fragment containing a hypervariable region.

6. The method for rapid preparation of an epidemic infectious bronchitis vaccine according to claim 5, wherein the S gene is replaced with one of S gene fragments of the epidemic infectious bronchitis virus strain or a fusion gene composed of multiple of the S gene fragments; and
the serotypes or genotypes of the epidemic infectious bronchitis virus strain are different from that of an infectious bronchitis virus H120 vaccine strain.

7. The method for rapid preparation of an epidemic infectious bronchitis vaccine according to claim 1, wherein the method comprises the following steps:
A1: conducting isolation to obtain two highly pathogenic infectious bronchitis epidemic wild virus strains of different serotypes or different genotypes, respectively extracting RNA and conducting RNA reverse transcription to obtain cDNA, and conducting amplification, by using each cDNA as a template, to correspondingly obtain an S2₁ gene and an S2₂ gene, and an S1₁ gene and an S1₂ gene that do not include the signal peptide region;
A2: fusing the S1₁ gene and S2₂ gene in step A1 or the S1₂ gene and S2₁ gene in step A1 to obtain a fusion gene S1₁+S2₂ or S1₂+S2₁;
A3: replacing the fusion gene S1₁+S2₂ or S1₂+S2₁ in step A2 on a constructed skeleton carrier of an H120 infectious clone by using a RED/ET technology and conducting screening to obtain a positive recombinant plasmid; and
A4: rescuing the positive recombinant plasmid in step A3 to obtain a recombinant virus that can immunize the two infectious bronchitis viruses in step A1.

8. The method for rapid preparation of an epidemic infectious bronchitis vaccine according to claim 7, wherein in step A3, the fusion gene S1₁+S2₂ is an S1 gene of GL15+ an S2 gene of GZ14, and the GenBank accession number of a GL15 sequence is KJ524616 and the GenBank accession number of a GZ14 sequence is KT946798.

9. The method for rapid preparation of an epidemic infectious bronchitis vaccine according to claim 7, wherein the S gene is replaced with one of S gene fragments of the epidemic infectious bronchitis virus strain or a fusion gene composed of multiple of the S gene fragments; and
the serotypes or genotypes of the epidemic infectious bronchitis virus strain are different from that of an infectious bronchitis virus H120 vaccine strain.

10. The method for rapid preparation of an epidemic infectious bronchitis vaccine according to claim 9, wherein the fusion gene is fused by the S1 gene of a serotype epidemic infectious bronchitis virus strain and an S2 gene of another serotype infectious bronchitis virus strain;
or the fusion gene is fused by the S1 gene of a genotype epidemic infectious bronchitis virus strain and an S2 gene of another genotype infectious bronchitis virus strain.

11. The method for rapid preparation of an epidemic infectious bronchitis vaccine according to claim 10, wherein the S2 gene is derived from the epidemic infectious bronchitis virus strain or an infectious bronchitis vaccine virus strain.

12. The method for rapid preparation of an epidemic infectious bronchitis vaccine according to claim 7, wherein the S1 gene is an S1 fragment containing a hypervariable region.

13. A method for rapid preparation of an epidemic infectious bronchitis vaccine, wherein an infectious clone of an infectious bronchitis virus (IBV) H120 vaccine strain is used as a skeleton carrier and an antigen gene in the skeleton carrier is replaced with a target antigen gene of an infectious bronchitis epidemic virus strain, to obtain a recombinant bronchitis virus; the target antigen gene and an N gene are simultaneously replaced; the target antigen gene is an S gene or an S1 gene; and a signal peptide region of the original S1 gene in the skeleton carrier is retained during the replacement.

14. The method for rapid preparation of an epidemic infectious bronchitis vaccine according to claim 13, wherein the S1 gene and an N gene are simultaneously replaced.

15. The method for rapid preparation of an epidemic infectious bronchitis vaccine according to claim 13, wherein the epidemic infectious bronchitis virus strain is a highly pathogenic wild virus strain.

16. The method for rapid preparation of an epidemic infectious bronchitis vaccine according to claim 14, wherein the S1 gene and N gene are derived from GL15 and the GenBank accession number of a GL15 sequence is KJ524616.
